(19) United States
(12) Patent Application Publication
(10) Pub. No.: US 2025/0195205 A1
(43) Pub. Date: Jun. 19, 2025

(54) REINFORCED MEDICAL IMPLANT AND METHOD OF USE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Cori G. Pierce, Salem, NH (US); Justin A. Callaway, Goffstown, NH (US); David A. Yeung, Andover, MA (US); Kevin M. Falco, Chelmsford, MA (US); Rachel S. Keen, Medford, MA (US); Shreya S. Medepalli, Chantilly, VA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/072,153

(22) Filed: Mar. 6, 2025

Related U.S. Application Data
(63) Continuation of application No. PCT/US2023/033001, filed on Sep. 18, 2023.
(60) Provisional application No. 63/408,278, filed on Sep. 20, 2022.

(51) Int. Cl. A61F 2/08; A61L 27/54

(52) U.S. Cl. CPC A61F 2/08; A61L 27/54

(57) ABSTRACT

Bio-compatible implants are used for treating soft tissue injuries. A bio-compatible implant may include a collagen scaffold and one or more reinforcing members that help to provide the bio-compatible implant with additional strength until such time as tissue grows into the implant, thereby reducing possible injuries that could otherwise be caused by an impatient patient. The reinforcing members may be bioabsorbable, for example.

### CROSS REFERENCE TO RELATED APPLICATIONS

This application is a continuation of International Application No. PCT/US2023/033001, filed on Sep. 18, 2023, which claims the benefit of and priority to U.S. Provisional Patent Application Ser. No. 63/408,278, filed on Sep. 20, 2022, the disclosures of which are incorporated herein by reference.

### TECHNICAL FIELD

The present disclosure pertains generally, but not by way of limitation, to orthopedic implants and methods of treatment. More particularly, the present disclosure relates to a tendon repair implant, such as one that is engineered for arthroscopic placement over or in the area of a full or partial thickness tear of the supraspinatus tendon of the shoulder

### BACKGROUND

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Adequate procedures do not exist for repairing a partial thickness tear of less than 50% in the supraspinatus tendon. Current procedures attempt to alleviate impingement or make room for movement of the tendon to prevent further damage and relieve discomfort but do not repair or strengthen the tendon. Use of the still damaged tendon can lead to further damage or injury. There is an ongoing need to deliver and adequately position medical implants during an arthroscopic procedure in order to treat injuries to the rotator cuff, rotator cuff tendons, or other soft tissue or tendon injuries throughout a body.

### BRIEF SUMMARY

This disclosure provides design, material, manufacturing method, and use alternatives for implants used for soft tissue repairs such as but not limited to rotator cuff repairs. An implant may include a collagen scaffold and one or more reinforcing members that help to provide the implant with additional strength until such time as tissue grows into the implant, thereby reducing possible injuries that could otherwise be caused by an impatient patient. The reinforcing members may be bioabsorbable, for example.

An example may be found in a bio-compatible implant that is adapted for use in repairing soft tissue damage. The bio-compatible implant includes a collagen scaffold having a major axis and a minor axis, the collagen scaffold defining a periphery, and a plurality of reinforcing fibers extending through the collagen scaffold, at least some of the plurality of reinforcing fibers at least substantially aligned with the major axis of the collagen scaffold. At least some of the reinforcing fibers extend beyond the periphery of the collagen scaffold.

Alternatively or additionally, the at least some of the reinforcing fibers extending beyond the periphery of the collagen scaffold may be adapted to be secured relative to a bone anchor.

Alternatively or additionally, at least some of the plurality of reinforcing fibers may include magnesium fibers.

Alternatively or additionally, the magnesium fibers may have an average diameter in a range of 0.1 to 1.5 microns.

Alternatively or additionally, the magnesium fibers may have an average length of up to about 50 millimeters.

Alternatively or additionally, the plurality of reinforcing fibers may be dispersed within the collagen scaffold.

Alternatively or additionally, at least some of the plurality of reinforcing fibers may be formed into a reinforcing structure.

Alternatively or additionally, the reinforcing structure may be encapsulated within the collagen scaffold.

Alternatively or additionally, the bio-compatible implant may further include a plurality of tie fibers extending through the collagen scaffold, at least some of the plurality of tie fibers at least substantially aligned with the minor axis of the collagen scaffold.

Another example may be found in a method of making a bio-compatible implant that includes a magnesium skeleton within a collagen substrate. The method includes forming a magnesium skeleton from a plurality of magnesium fibers and contacting the magnesium skeleton with a collagen solution. The collagen solution on the magnesium skeleton is allowed to dry in order to encapsulate the magnesium skeleton within a collagen substrate.

Alternatively or additionally, forming the magnesium skeleton may include bundling together a plurality of magnesium fibers.

Alternatively or additionally, forming the magnesium skeleton may include forming the magnesium skeleton from a first plurality of magnesium fibers having an average length shorter than a long dimension of the resulting bio-compatible implant and a second plurality of magnesium fibers having an average length greater than the long dimension of the resulting bio-compatible implant.

Alternatively or additionally, at least some of the magnesium fibers of the second plurality of magnesium fibers may be adapted to secured relative to a bone anchor.

Alternatively or additionally, contacting the magnesium skeleton with the collagen solution may include immersing the magnesium skeleton into the collagen solution.

Alternatively or additionally, contacting the magnesium skeleton with the collagen solution may include repeatedly dipping the magnesium skeleton into the collagen solution.

Alternatively or additionally, contacting the magnesium skeleton with the collagen solution may include repeatedly spraying the magnesium skeleton with the collagen solution.

Another example may be found in a bio-compatible implant adapted for use in repairing soft tissue damage. The bio-compatible implant includes a collagen scaffold including a plurality of non-woven reconstituted collagen fibers and a reinforcing sheet that is coupled with the collagen scaffold, the reinforcing sheet including a plurality of reinforcing fibers extending longitudinally within the reinforcing sheet along a load axis of the bio-compatible implant.

Alternatively or additionally, at least some of the reinforcing fibers may include magnesium fibers.

Alternatively or additionally, the magnesium fibers may have an average length up to about 50 millimeters and/or an average diameter in a range of 0.1 microns to 1.5 microns.

Alternatively or additionally, at least some of the reinforcing fibers may include elastin fibers.

Alternatively or additionally, at least some of the elastin fibers may be randomly dispersed within the collagen scaffold.

Alternatively or additionally, the elastin fibers may have an average length in a range of 10 microns to 500 microns and/or an average diameter in a range of 0.1 microns to 1.5 microns.

Alternatively or additionally, the reinforcing sheet may further include a plurality of collagen fibers.

Alternatively or additionally, the reinforcing sheet may further include a plurality of high-density, highly cross-linked collagen fibers.

Alternatively or additionally, the bio-compatible implant may further include a plurality of tie fibers extending through the reinforcing sheet, at least some of the plurality of tie fibers at least substantially non-parallel with the load axis of the bio-compatible implant.

Another example may be found in a bio-compatible implant that is adapted for use in repairing soft tissue damage. The bio-compatible implant includes a first collagen layer, a second collagen layer, and a reinforcing silk layer that is disposed between the first collagen layer and the second collagen layer.

Alternatively or additionally, the bio-compatible implant may have an initial tensile modulus of 6 to 12 mega Pascals (MPa) and an ultimate tensile strength of at least about 50 Newtons (N).

Alternatively or additionally, the reinforcing silk layer may include a plurality of silk fibers.

Alternatively or additionally, the reinforcing silk layer may include a plurality of electro-spun silk fibers.

Alternatively or additionally, the reinforcing silk layer may include a porous structure.

Alternatively or additionally, at least part of the first collagen layer may penetrate into the porous structure.

Alternatively or additionally, at least part of the second collagen layer may penetrate into the porous structure.

Alternatively or additionally, the first collagen layer and the second collagen layer together may encapsulate the reinforcing silk layer.

Alternatively or additionally, the reinforcing silk layer may further include a plurality of chitosan fibers.

Alternatively or additionally, the bio-compatible implant may have a thickness that is in a range of 1 to 3 millimeters.

Alternatively or additionally, the bio-compatible implant may have a length that is in a range of 25 to 45 millimeters and a width that is in a range of 20 to 30 millimeters.

Another example may be found in a kit for use in repairing soft tissue damage. The kit includes a bio-compatible implant that is adapted for repairing soft tissue damage and an applicator. The bio-compatible implant includes a collagen scaffold defining a first primary surface and a second primary surface, with a periphery extending around the collagen scaffold between the first primary surface and the second primary surface, with a plurality of defined spaces relative to the collagen scaffold for receiving a strengthening agent after the bio-compatible implant has been deployed at a treatment site. The applicator includes the strengthening agent and is adapted to allow a user to apply the strengthening agent to at least some of the plurality of defined spaces relative to the collagen scaffold after the bio-compatible implant has been deployed at the treatment site.

Alternatively or additionally, at least some of the plurality of defined spaces relative to the collagen scaffold may include voids formed within the first primary surface.

Alternatively or additionally, at least some of the plurality of defined spaces relative to the collagen scaffold may include voids extending through an interior of the collagen scaffold and reaching the periphery of the collagen scaffold.

Alternatively or additionally, the strengthening agent may be adapted to solidify upon curing post-application.

Alternatively or additionally, the strengthening agent may include a salt solution that flows into the plurality of defined spaces and subsequently solidifies.

Alternatively or additionally, the kit may further include a patterned guide that may be adapted to be placed in contact over either the first primary surface or the second primary surface, the patterned mold including cutouts that guide placement of the strengthening agent.

Alternatively or additionally, the patterned guide may include polytetrafluoroethylene.

Another example may be found in a bio-compatible implant adapted for use in repairing soft tissue damage. The bio-compatible implant includes a collagen scaffold having a major axis and a minor axis, the collagen scaffold defining a periphery, and one or more coiled reinforcing members that extend through the periphery of the collagen scaffold.

Alternatively or additionally, the one or more coiled reinforcing members may be adapted to stretch up to ten percent of their length.

Alternatively or additionally, the one or more coiled reinforcing members may be adapted to be secured relative to a bone anchor.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates a cross-section of an anterior view of a shoulder of a patient;

FIG. 2 illustrates a shoulder including a head of the humerus mating with the glenoid fossa of the scapula at a glenohumeral joint and an implant affixed to a tendon;

FIG. 3A illustrates an example implant delivery device attached to an implant;

FIG. 3B illustrates an example delivery device attached to an implant;

FIG. 3C illustrates an example delivery device attached to an implant;

FIG. 4 illustrates an example implant delivery device attached to an implant;

FIG. 5 shows an example implant;

FIGS. 6A through 6D show schematic cross-sections along a line 6-6 of FIG. 5;

FIGS. 7A and 7B show example patterns for a reinforcing member;

FIGS. 8A and 8B show example patterns for a reinforcing member;

FIG. 9 shows an example implant;

FIG. 10 is a cross-sectional view taken along a line 10-10 of FIG. 9;

FIG. 11 shows an example implant;

FIG. 12 is a cross-sectional view taken along a line 12-12 of FIG. 11;

FIG. 13 shows an example implant;

FIG. 14 is a cross-sectional view taken along a line 14-14 of FIG. 13;

FIG. 15 shows an example implant;

FIG. 16 shows an example kit for implanting an implant;

FIG. 17 shows an example implant; and

FIG. 18 shows an example implant.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

With its complexity, range of motion and extensive use, a common soft tissue injury is damage to the rotator cuff or rotator cuff tendons. Damage to the rotator cuff is a potentially serious medical condition that may occur during hyperextension, from an acute traumatic tear or from overuse of the joint. Current repair procedures may attempt to alleviate impingement or make room for movement of the tendon to prevent further damage and relieve discomfort but do not repair or strengthen the tendon. An accepted treatment for rotator cuff tears may include reattaching the torn tendon to the humeral head using sutures. Additionally, in treating rotator cuff tears, an accepted practice may also include the placement of a scaffold over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

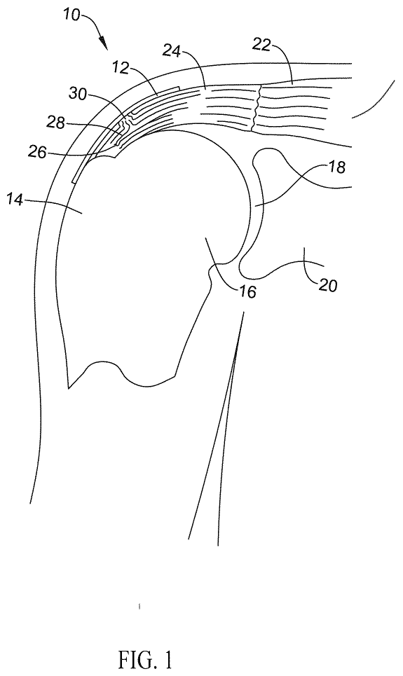

FIG. 1 shows a cross-sectional view of a shoulder 10 including an example implant 12. Shoulder 10 further shows a head 14 of a humerus 16 mating with a glenoid fossa 18 of scapula 20. The glenoid fossa 18 comprises a shallow depression in scapula 20. A supraspinatus tendon 22 is also shown. These muscles (along with others) control the movement of the humerus 16 relative to the scapula 20. A distal tendon 24 of the supraspinatus tendon 22 meets the humerus 16 at an insertion point 26.

In FIG. 1, the distal tendon 24 includes a damaged portion 28 located near the insertion point 26. The damaged portion 28 includes a tear 30 extending partially through the distal tendon 24. The tear 30 may be referred to as a partial thickness tear. The depicted partial thickness tear 30 is on the bursal side of the tendon, however, the tear may also be on the opposite or articular side of the distal tendon 24 and/or may include internal tears to the distal tendon 24 not visible on either surface. In other instances the tear 30 may be a full thickness tear.

FIG. 1 further illustrates that the tendon repair implant 12 has been placed over the partial thickness tear 30. In this example, the tendon repair implant 12 is placed on the bursal side of the tendon regardless of whether the tear is on the bursal side, articular side or within the tendon. Further, the tendon repair implant 12 may overlay multiple tears.

Although one possible treatment site is described in the figures as being located in a shoulder joint, the implant 12 may be used at a variety of different treatment sites, such as the hip, knee, ankle, et. Furthermore, the implant 12 may be used for any of a variety of soft tissue repairs, such as but not limited to the Gluteus Medius, which is a large fan-shaped muscle located in the posterior hip, the Hip Capsule, which is also in the hip. The implant 12 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The implant 12 may be used in treating soft tissue injuries in the Achilles tendon. The implant 12 may be used in treating soft tissue injuries for any areas of the body that will accommodate the implant 12.

In some instances, delivery of an implant 12 (e.g., a sheet-like implant) to a target site of a patient may require a physician to create an incision in the patient sufficient to access the target implant site. After creating this “access site,” the physician may insert an implant delivery system through the access site and position the distal end of the implant delivery system adjacent the target implant site. The physician may then manipulate the implant delivery system to deploy an implant out of a delivery sheath adjacent the target implant site.

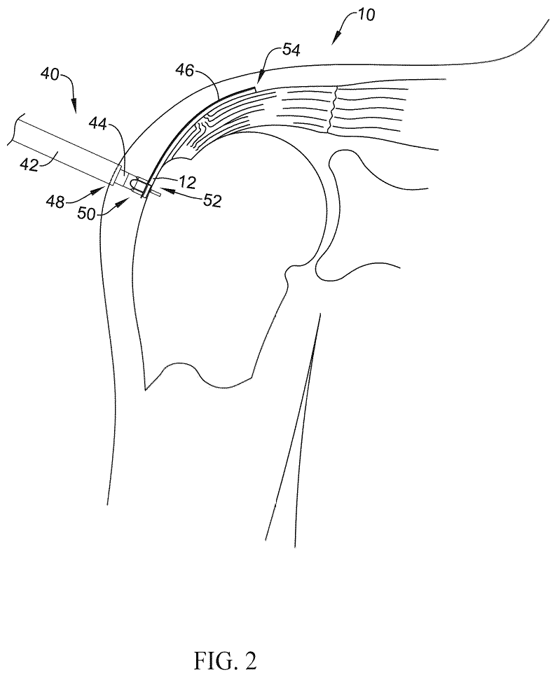

For example, FIG. 2 provides a perspective view of an implant delivery system 40 extending through the shoulder 10 of a patient. FIG. 2 the shows implant delivery system 40 deployed adjacent a target site (e.g., a tear in the supraspinatus tendon). In at least some embodiments, the implant delivery system 40 includes a sheath member 42 (e.g., a cannula) including a proximal portion (not shown), a distal portion 48 and a lumen extending within at least a portion of the cannula 42. Further, the implant delivery system 40 may include a delivery shaft 44 extending within the lumen of the sheath member 42 and longitudinally movable relative thereto.

The delivery shaft 44 may include a proximal portion (not shown) extending out of the proximal portion of the sheath member 42 and/or otherwise manipulatable relative to the sheath member 42 by a user. Additionally, in some examples the proximal portion of the delivery shaft 44 and/or the sheath member 44 may be coupled to a handle member (not shown). The handle member may be utilized to manipulate the delivery shaft 44. For example, the handle member may be utilized to impart a rotational force to the delivery shaft 44.

In addition, the delivery shaft 44 may include a distal portion 50 extending out of the distal portion 48 of the sheath member 42. Further, the delivery shaft 44 may include a lumen extending therein. The lumen of the delivery shaft 44 may extend along a portion or the entire length of the delivery shaft 44 (e.g., from the distal portion 50 to the proximal portion of the delivery shaft 44).

The delivery system 40 may further include a detachable frame member 46 attached to the distal portion 50 of the delivery shaft 44. As shown in FIG. 2, the detachable frame 46 may be attached to an implant 12 (e.g., a sheet-like implant). For purposes of the discussion herein, the combined structure including the frame 46 and the implant 12 may be defined as having a proximal end 52 and a distal end 54 as illustrated in FIG. 2.

When initially positioning the frame 46 and implant 12 adjacent a target site, a clinician may orient the frame 46 and the implant 12 (for example, via a handle member attached to a proximal portion of the delivery shaft 44) such that the proximal portion 52 may be adjacent (e.g., overlaid) on a portion of the humerus (e.g., on the bone), while the distal portion 54 of the frame 46 and the implant 12 may overlay the distal tendon 24.

As described above, delivery of implant delivery system 40 may include the insertion of delivery sheath 42 through an access site (e.g., incision) and advancement to a target site. After positioning the distal end 48 of the delivery sheath 42 proximate the target site, a clinician may deploy the detachable frame 46 in combination with the implant 12 out of the lumen located within and along the distal portion 48 of the delivery sheath 42, such as by retracting the delivery sheath 42 relative to the delivery shaft 44 and the frame 46, and positioning the implant 12 and the frame 46 over the target site.

Prior to deployment, the detachable frame 46 and the implant 12 combination may be contained (e.g., housed) within the lumen of the delivery sheath 42 for subsequent deployment distally out distal opening of the delivery sheath 42. As will be described in greater detail below, the combination of the detachable frame 46 and the implant 12 may wrap and/or fold upon itself such that it may be positioned within the lumen of the delivery sheath 42. Alternatively, the detachable frame 46 and the implant 12 may warp and/or fold around the implant delivery shaft 44 while disposed within the delivery sheath 42.

In some cases, the detachable frame 46 may be formed as a monolithic structure by being formed (e.g., machined, cut, shaped, stamped, laser-cut, etc.) as a unitary structure from a single piece of material. It is contemplated that detachable frame 46 may be constructed using alternative materials and/or manufacturing methodologies. For example, the frame 46, or portions thereof, may be constructed from a polymeric material, a ceramic material and/or other various materials. Additionally, the frame 46 may be manufactured via an injection molding or alternative polymer manufacturing methodologies. Alternatively, the frame 46 may be formed through a 3-D printing process, if desired. Further, different portions of the frame 46 may be made from a variety of materials and combined using alternative methodologies. Variations of combining different materials with different portions of the frame 46 are contemplated.

For simplicity purposes, when combined with an example implant 12, the frame 46 may be defined as having a first surface that faces away from the implant 12 when the implant 12 is attached to the frame 46 (e.g., a first surface that faces away from a target site in the body) and a second surface that faces the example implant 12 (e.g., a second surface that faces a target site in the body). In some instances, attachment apertures 70 may extend from the first surface to the second surface. In other words, in some instances, attachment apertures 70 may be defined as holes and/or openings that extend through the thickness of the frame 46 from the first surface of the frame 46 that faces away from the implant 12 to the second surface of the frame 46 that faces toward the implant 12.

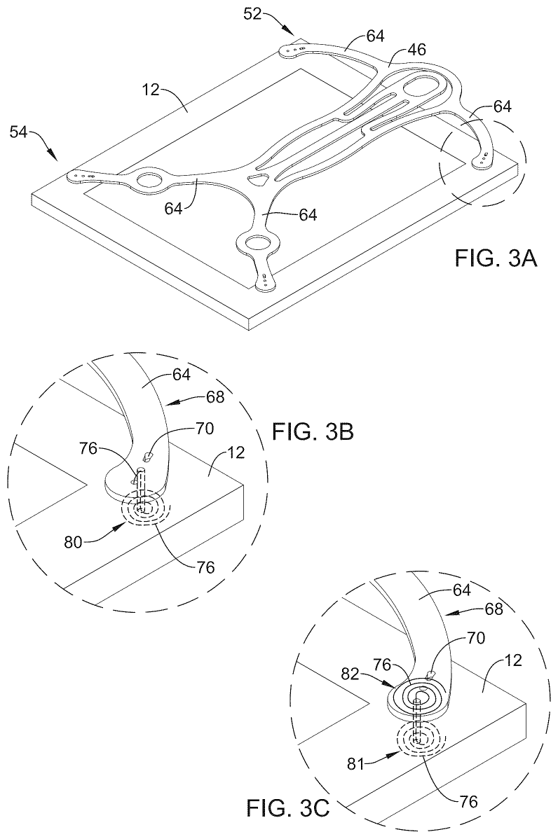

Attachment apertures 70 may be utilized to attach and/or couple frame 46 to an example implant 12. FIG. 3A shows an example frame 46 attached to an example implant 12. Further, FIG. 3 shows example frame 46 attached to example implant 12 at the distal or free end of each of four attachment arms 64, respectively. Attachment of free distal ends of attachment arms 64 to implant 12 may be made by any desired attachment mechanism.

FIG. 3B shows a detailed view of a portion of the proximal portion 54 of a frame 46 attached to an implant 12. Further, FIG. 3B shows example attachment arm 64 including a distal portion 68. Three attachment apertures 70 are positioned along the distal portion 68 of the attachment arm 64. Additionally, FIG. 3B shows an example attachment member (e.g. wire) 76 extending between and through one or more of the attachment apertures 70 located on the distal portion 68 of the attachment arms 64.

The attachment members 76 may be one of several structures and/or techniques contemplated to attach the example frame 46 to the example implant 12. As shown in FIG. 3B, the attachment member 76 may be positioned, looped, wound and/or threaded through one or more attachment apertures 70 such that the attachment member 76 is prevented from being pulled away from the distal portion 68 of the attachment arm 64. In other words, winding the attachment member 76 through one or more of the attachment apertures 70 may effectively affix the attachment member 76 onto the attachment arm 64. In other words, it is contemplated that the attachment member 76 may be affixed to the distal portion 68 of the attachment arms 64 (via attachment apertures 70, for example) without having either end of the attachment member 76 directly attached (e.g., welded, tied, etc.) to any structure (e.g., frame 46). In some instances, the attachment member 76 may be wrapped and/or looped through the attachment apertures 70 one or more times to provide a friction fit and/or resistive tension to unraveling or unwinding as a withdrawal force is applied to the attachment member 76. In other instances, the implant may be attached to the attachment arms 64 in another fashion, such as with other attachment members attached to and/or extending from the attachment arms 64.

While FIG. 3B shows a single attachment member 76 extending between two attachment apertures 70, it is contemplated that the attachment member 76 may extend and/or wrap between two or more of the attachment apertures 70. For example, it is contemplated that the attachment member 76 may be woven (e.g., over-and-under) through three apertures 70 in order to lock the attachment member 76 to the distal end 68 of the attachment arm 64.

The above discussion and the forgoing examples are not intended to limit the disclosure to using an attachment member (e.g., wire, thread, cable, etc.) to attach the frame 46 to the implant 12. Rather, a variety of methodologies may be utilized to attach the frame 46 to the implant 12. For example, adhesives may be used alone or in combination with another attachment mechanism to attach the frame 46 to the implant 12. Additionally, a variety of injection molding techniques may be employed to attach the frame 46 to the implant 12. Further, combinations of the disclosed techniques may be used to attach the frame 46 to the implant 12. For example, an attachment member 76 may be used in conjunction with an adhesive to attach the frame 46 to the implant 12 without having to wind the attachment member 76 through the attachment apertures 70.

As stated above, it is contemplated in the examples discussed herein that the frame 46 may be able to be “detached” from the implant 12. For example, the frame 46 may be configured to detach from the implant 12 after the implant 12 has been affixed to a target site in the body. Therefore, it can be appreciated that in some examples disclosed herein, the frame member 46 may be temporarily attached to the implant 12. For example, the frame member 46 may be coupled, affixed or attached to the implant 12 while positioned within the delivery sheath 42, deployed out of the delivery sheath 42 and maneuvered into position relative to a target site. Once positioned at the target site (e.g., along the tendon and/or humeral head), the implant 12 may be affixed to the target site via bio-adhesives with or without the use of tissue anchors and/or sutures, as will be discussed. However, once the implant 12 has been affixed to the target site, the frame 46 may be pulled away (e.g., detached) from the implant 12 and removed from the body.

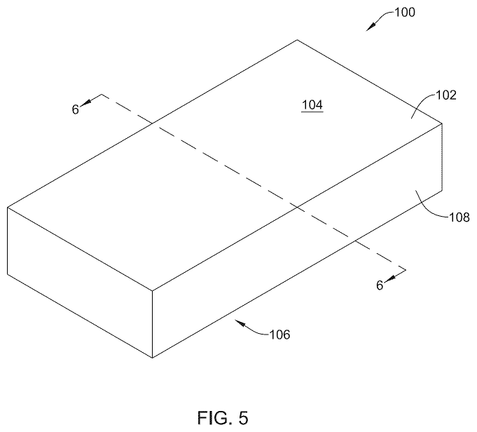

FIG. 3B shows an example attachment configuration which may allow the frame 46 to detach from the implant 12. FIG. 5B shows attachment member 76 wound in a spiral pattern 80 along the surface of implant 12 facing a target site. In other words, attachment member 76 may form a spiral pattern 80 that remains in a plane substantially parallel to the plane of the surface of implant 12 which faces a target site. Further, it can be appreciated that attachment member 76 may extend from the side of attachment arm 64 facing away from implant 12, through the combined thickness of the attachment arm 64 and implant 12, eventually exiting implant 12 on the surface of implant 12 facing a target site. Further, it can be appreciated that the spiral pattern 80 shown in FIG. 5B is one of a variety of configurations for which attachment member 76 may be wound in order to prevent frame 46 from prematurely releasing from implant 12.

Attachment member 76 may have a first end secured to a free distal end of attachment arm 64 positioned on a first side of implant 12 and have a second end positioned on a second, opposite side of implant 12. In some instances, attachment member 76 may extend through implant 12 from the first side of implant 12 to the second side of implant 12. However, in other instances, attachment member 76 may extend around an edge of implant 12 from the first side of implant 12 to the second side of implant 12.

In some cases, the attachment member 76 may be configured to be detached from implant 12 upon application of a threshold level of force. For example, the spiral pattern 80 shown in FIG. 3B may provide the frame 46 the ability to detach from the implant 12 when a threshold “pull-away force” is applied to the frame 46. For example, after the implant 12 is affixed to a target site, a clinician may apply a force to the frame 46 (via a tether, for example) such that the frame 46 is pulled away from the implant 12. Provided the force is great enough (e.g., the threshold force is met), the attachment members 76 (e.g., spiral portion 80 of the attachment member 76 shown in FIG. 3B) may be unwound and pulled back through the “body” (e.g., thickness) of the implant 12, thereby releasing the frame 46 from the implant 12. In other words, provided a threshold pull-away force is applied to the frame 46, the attachment member 76 forming the spiral 80 shown in FIG. 3B may unwind and pull back through the implant 12.

FIG. 3C shows another example method to attach the frame 46 to an example implant 12. As shown in FIG. 3C, the attachment member 76 may include a spiral 81 positioned on the surface of the implant 12 which faces away from a target site (similar to the spiral 80 shown in FIG. 3B). Additionally, FIG. 3C shows that the attachment member 76 may include a second spiral 82 positioned on the surface of an attachment arm 68 that faces away from the implant 12. In other words, FIG. 3C shows two spirals 81/82 formed at opposite ends of the attachment member 76 and positioned on both the attachment arm 64 facing away from the implant 12 (e.g., spiral 82 of FIG. 5C) and on the side of the implant 12 lying along a treatment site (e.g., spiral 81 of FIG. 3C). The configuration of spirals 81/82 may provide a frame 46 with a “releasable” connection to the implant 12 similar to that discussed with respect to FIG. 3B.

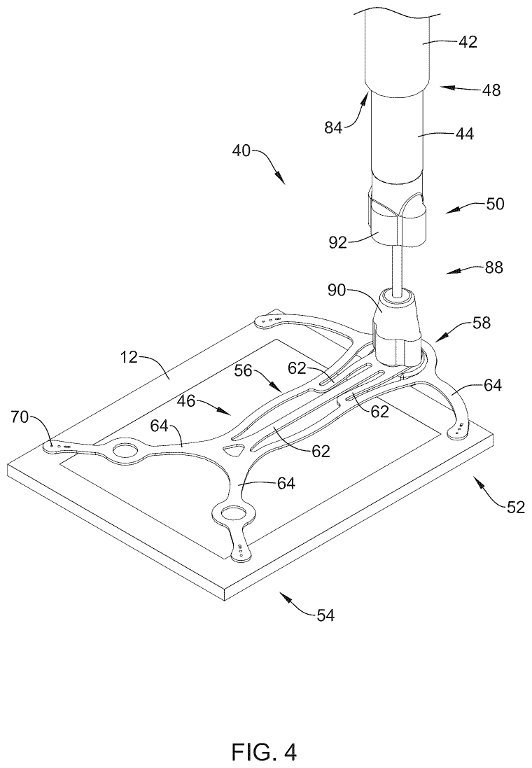

FIG. 4 shows an example frame 46 coupled to an example implant 12 via the attachment members 76 as described above. Further, FIG. 4 shows the frame 46 in combination with the implant 12 coupled to an example implant delivery system 40. Similar to that discussed with respect to FIG. 2, the implant delivery system 40 includes implant delivery shaft 44 extending through an example lumen 84 of an example delivery sheath 42.

Further, FIG. 4 shows the delivery shaft 44 coupled to the frame 46 via a connection assembly 88. The connection assembly 88 may include a first connection member 90 attached to the head portion 58 of the frame 46 and a second connection member 92 attached to the distal end 50 of the delivery shaft 44. While FIG. 4 does not directly show the first connection member 90 attached directly to the second connection member 92, it can be appreciated that the first and second connection members 90/92 of connection assembly 88 may form a mating connection. For example, in some instances, the first connection member 90 may form a male connection member while the second connection member 92 may form a mating female connection member. In other words, in some examples the second connection member 92 may include a cavity which is configured to extend over and allow the first connection member 90 to be inserted therein. In other instances, the first connection member 90 may be a female connection member, while the second connection member 92 may be a mating male connection member.

Additionally, as shown in FIG. 4, it is contemplated that second connection member 92 may disengage or decouple from the first connection member 90. For example, in some instances the connection assembly 88 (including first and second connection members 90/92) may be defined as a “quick release” connection assembly, or otherwise decoupling connection assembly. It is further contemplated that a variety of design configurations may be employed to engage/disengage (i.e., couple/decouple) the first and second connection members 90/92 from one another. For example, the first and second connection members 90/92 may be coupled via a threaded connection, friction fit, spring loaded connection, bayonet connection, movable collar or other actuation mechanism, or the like. Further, the connection member 90/92 may be engaged/disengaged by an operator of the device.

In some instances, the delivery shaft 44 may be attached (via the connection assembly 88, for example) to the head portion 58 of the frame member 46. As shown in FIG. 4, the first connection member 90 of the connection assembly 88 may attach to head portion 58 via an aperture 60 (not shown). In some instances, the first connection member 90 may be attached to the head portion 58 of the frame member 46 via a variety of mechanical fastening means (e.g., injection molding, encapsulation, bonding, etc.)

As discussed above, in some instances, a physician may insert the implant delivery system 40 (including the delivery sheath 42, the delivery shaft 44, the frame 46 and the implant 12) through an incision and position the distal end of the implant delivery system 40 adjacent a target implant site (e.g., torn tendon). Once adjacent the target site, the physician may manipulate the implant delivery shaft 44 to advance the implant 12 (while attached to the detachable frame 46) out of the delivery sheath 42 adjacent the target implant site. For example, the physician may retract the delivery sheath 42 proximally relative to the delivery shaft 44 and the frame 46 and/or may advance the delivery shaft 44 and the frame 46 distally relative to the delivery sheath 42.

FIG. 4 shows the frame 46 and the implant 12 deployed from the distal portion 48 of the delivery sheath 42. In some instances, the frame 46 and the implant 12 may have a substantially concave shape with respect to the delivery sheath 42. It can be appreciated that the concave shape of the frame member 46 and the implant 12 may facilitate positioning the implant 12 along the generally rounded shape of the human shoulder.

However, when positioned in the delivery sheath 42 (e.g., prior to deployment) the frame 46 and the implant 12 may be wrapped around the delivery shaft 44 in a convex configuration. Therefore, the frame 46 and the implant 12 may shift from a first convex configuration (while wrapped tightly around the delivery shaft 44 within the lumen 84 of the delivery sheath 42) to a second concave configuration when advanced (e.g., deployed) out of the sheath 42.

In other words, the frame 46 and the implant 12 may be attached to the deliver shaft 44 via the connection assembly 88 when positioned within the lumen 84 of the delivery sheath 42. In one example, when positioned within the delivery sheath 42, the frame 46 and implant 12 may wrap, or extend around, the delivery shaft 44. The position of the frame 46 and implant 12 may be in a convex configuration with respect to the distal end 50 of the delivery shaft 44. As the frame 46 and implant 12 are deployed out of the distal end 50 of the delivery shaft 44, the frame 46 and implant 12 may “shift” from a convex configuration to a concave configuration (as viewed with respect to the distal end 50 of delivery shaft 44). Additional details regarding the frame 46 and the implant delivery system 40 may be found in U.S. Pat. No. 10,314,689, which is incorporated by reference herein in its entirety.

FIG. 5 is a schematic diagram of an example bio-compatible implant 100. The bio-compatible implant 100 may be used for any of a variety of soft tissue repairs, such as but not limited to the Gluteus Medius, which is a large fan-shaped muscle located in the posterior hip, the Hip Capsule, which is also in the hip. The bio-compatible implant 100 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 100 may be used in treating soft tissue injuries in the Achilles tendon. The bio-compatible implant 100 may be used in treating soft tissue injuries for any areas of the body that will accommodate the bio-compatible implant 100.

For purposes of illustration, the bio-compatible implant 100 will be described herein with respect to rotator cuff repairs. The bio-compatible implant 100 may be considered as being an example of the implant 12 discussed with respect to FIGS. 1-2, 3A-3C and 4. The bio-compatible implant 100 may be considered as including a scaffold 102. In some instances, the scaffold 102 may define a first primary surface 104 and a second primary surface 106, and a periphery 108 that extends around between the first primary surface 104 and the second primary surface 106.

While drawn as rectilinear, it will be appreciated that this is merely illustrative, as the bio-compatible implant 100 may take any desired shape to best fit at a desired treatment site such as but not limited to a rotator cuff repair. In some cases, the bio-compatible implant 100 may be largely rectilinear, with rounded over corners, for example. In some cases, the bio-compatible implant 100 may be at least partially ovoid. The first primary surface 104 and the second primary surface 106 may be planar or curved. In some cases, the bio-compatible implant 100 may curve around a treatment site. These are just examples.

In some cases, the scaffold 102 may be a collagen scaffold, although other materials are contemplated. In some cases, the scaffold 102 is formed of collagen that has been dehydrated. The scaffold 102 may be formed as a fibrous collection of collagen fibers that define a porous scaffold with a large number of void spaces.

The collagen used to form the scaffold 102 may come from any of a variety of different sources. Collagen is a main structural protein in the extracellular matrix found in various connective tissues and thus can be obtained from various animals. For example, the collagen used to form the scaffold 102 may be bovine-based, i.e., from cows. In some cases, the collagen used to form the scaffold 102 may come from bovine tendon material. As an illustrative but non-limiting example, bovine Achilles tendon is digested down to highly purified collagen, and is reconstituted into a sheet by spinning the fibers around a mandrel. The scaffold 102 may be formed of collagen that has been obtained in other methods as well.

While the scaffold 102 may be formed having a variety of different porosity levels (defined as relative amount of void space to solid material), in some cases the scaffold 102 may have a porosity of at least 50 percent or more, at least 60 percent or more, at least 70 percent or more, or at least 80 percent or more. In some cases, the scaffold 102 may have a porosity of 60 percent to 90 percent, 70 percent to 90 percent, 80 percent to 90 percent, or 85 percent to 90 percent.

Another way to define the scaffold 102 is in terms of average pore size. Pore size refers to a diameter of voids, or empty spaces, formed within the scaffold 102. Average pore size, accordingly, refers to an average diameter of these voids. In some cases, the scaffold 102 may have an average pore size of 20 microns or greater, or 30 microns or greater, or 40 microns or greater, or 50 microns or greater, or 60 microns or greater, or 70 microns or greater, or 80 microns or greater, or 90 microns or greater. In some cases, the scaffold 102 may have an average pore size that is in a range of 100 microns to 500 microns, 100 microns to 400 microns, 100 microns to 300 microns, 100 microns to 150 microns, or 200 microns to 400 microns, for example.

While not expressly shown in FIG. 5, the bio-compatible implant 100 may include a strengthening or reinforcing element that is adapted to provide strength to the soft tissue repair until tissue ingrowth into the bio-compatible implant 100 has occurred. It will be appreciated that the ultimate goal of the bio-compatible implant 100 is to facilitate tissue growth that provides long term strength and functionality to the soft tissue repair. The strengthening or reinforcing element, which may be biosorbable, is merely intended to provide additional strength to the bio-compatible implant 100 immediately after implantation of the bio-compatible implant 100. As the soft-tissue repair gains strength as a result of tissue ingrowth, the strengthening or reinforcing element becomes less important and in some cases may dissolve away.

The bio-compatible implant 100 may be dimensioned in accordance with its desired use. For example, if the bio-compatible implant 100 is intended for use in a rotator cuff repair, the bio-compatible implant 100 may have an overall length of 25 to 45 millimeters (mm) and an overall width of 20 to 30 mm. If the bio-compatible implant 100 is intended for use in an Achilles tendon repair, the bio-compatible implant 100 may have an overall length that is greater than 25 to 45 mm and an overall width that is less than 20 to 30 mm. In some cases, the bio-compatible implant 100 may have an overall thickness that is 1 to 3 mm. In some cases, the bio-compatible implant 100 may have an overall thickness that is about 2 mm. In defining these dimensions, it will be appreciated that the collagen scaffold 102 may not have a uniform thickness throughout the collagen scaffold 102, and the perimeter 108 may not be as evenly defined as shown in the rectilinear representation of FIG. 5. These dimensions may be considered as being averages, for example.

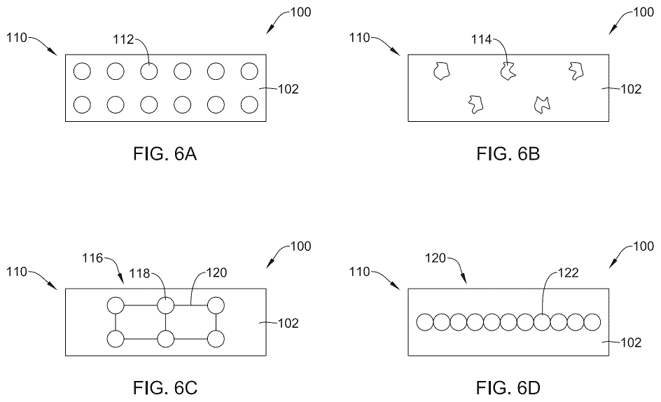

FIGS. 6A through 6D are schematic cross-sectional views of the bio-compatible implant 100, taken along the line 6-6 of FIG. 5, showing several examples of possible reinforcing elements 110. In some cases, the reinforcing elements 110 may be inserted into the collagen scaffold 102 after formation of the collagen scaffold 102. In some instances, the reinforcing elements 110 may be encapsulated within the collagen scaffold 102 by forming the collagen scaffold 102 around the reinforcing elements 110. As an example, the reinforcing elements 110 may be immersed into a solution of collagen. The reinforcing elements 110 may be repeatedly dipped into a solution of collagen, thereby building up the collagen scaffold 102. In some instances, the reinforcing elements 110 may be repeatedly sprayed onto the reinforcing elements, thereby building up the collagen scaffold 102.

In FIG. 6A, for example, the reinforcing element 110 includes a plurality of elongate members 112 that are dispersed within the collagen scaffold 102. In some instances, at least some of the elongate members 112 are oriented such that the elongate members 112 are at least substantially aligned with a longitudinal or load axis of the bio-compatible implant 100. In this, substantially aligned may describe an alignment within ten degrees of parallel, or perhaps within twenty degrees of parallel. The elongate members 112 may be organized in a symmetric fashion, as shown. In some cases, the elongate members 112 may be more randomly distributed within the collagen scaffold 102.

While FIG. 6A shows a total of twelve elongate members 112, this is merely illustrative. In some cases, the bio-compatible implant 100 may include one, two, three, four, five, six, seven, eight, nine, ten or eleven elongate members 112. The bio-compatible implant 100 may include thirteen, fourteen, fifteen or even more elongate members 112, for example.

The elongate members 112 may take any of a variety of different forms. In some cases, as shown, each of the elongate members 112 may have a circular or at least substantially circular cross-sectional profile. In some instances, at least some of the elongate members 112 may be fibers that are formed of a biosorbable material. At least some of the elongate members 112 may be formed of a biosorbable polymer. At least some of the elongate members 112 may be formed of a biosorbable metal. As an example, at least some of the elongate members 112 may be magnesium fibers. As another example, at least some of the elongate members 112 may be elastic fibers. In some cases, at least some of the elongate members 112 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the elongate members 112 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers may have a length that is greater than the overall length of the bio-compatible implant 100 such that at least some of the magnesium fibers extend beyond the periphery 108 of the bio-compatible implant 100. In some cases, the magnesium fibers that extend beyond the periphery 108 of the bio-compatible implant 100 may be used to help anchor the bio-compatible implant 100 at the treatment site by engaging one or more of the magnesium fibers with a bone anchor such as a bone staple. Other anchoring devices are also contemplated.

In instances in which at least some of the elongate members 112 are elastin fibers, the elastin fibers may have average diameters that are in a range of 0.1 microns to 1.5 microns, for example. At least some of the elastin fibers may have a circular cross-sectional profile, as shown. In some cases, at least some of the elastin fibers may have a less-defined cross-sectional profile. It will be appreciated that elastin is a naturally occurring, globular protein, and it may be difficult to ascertain exact dimensions. In some cases, elastin fibers may sprawl in both a longitudinal direction and in a radial direction within the collagen scaffold 102. The elastin fibers may have lengths that range from 10 microns to 500 microns, for example. The elastin fibers may have an average length that is less than an overall length of the bio-compatible implant 100. In some cases, at least some of the elastin fibers may have a length that is greater than the overall length of the bio-compatible implant 100 such that at least some of the elastin fibers extend beyond the periphery 108 of the bio-compatible implant 100. In some cases, the elastin fibers that extend beyond the periphery 108 of the bio-compatible implant 100 may be used to help anchor the bio-compatible implant 100 at the treatment site by engaging one or more of the elastin fibers with a bone anchor such as a bone staple. Other anchoring devices are also contemplated.

In instances in which at least some of the elongate members 112 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 100. In some cases, at least some of the high density, highly cross-linked collagen fibers may have a length that is greater than the overall length of the bio-compatible implant 100 such that at least some of the high density, highly cross-linked collagen fibers extend beyond the periphery 108 of the bio-compatible implant 100. In some cases, the high density, highly cross-linked collagen fibers that extend beyond the periphery 108 of the bio-compatible implant 100 may be used to help anchor the bio-compatible implant 100 at the treatment site by engaging one or more of the high density, highly cross-linked collagen fibers with a bone anchor such as a bone staple. Other anchoring devices are also contemplated.

In FIG. 6B, the reinforcing element 110 includes a plurality of elongate members 114 that are dispersed within the collagen scaffold 102. In some instances, at least some of the elongate members 114 are oriented such that the elongate members 114 are at least substantially aligned with a longitudinal or load axis of the bio-compatible implant 100. In this, substantially aligned may describe an alignment within ten degrees of parallel, or perhaps within twenty degrees of parallel. The elongate members 114 may be organized in a symmetric fashion, or may be more randomly distributed within the collagen scaffold 102.

While FIG. 6B shows a total of five elongate members 114, this is merely illustrative. In some cases, the bio-compatible implant 100 may include one, two, three, four, six, seven, eight, nine, ten, eleven, twelve, thirteen, fourteen, fifteen or even more elongate members 114, for example.

The elongate members 114 may take any of a variety of different forms. In some cases, as shown, each of the elongate members 114 may have a random cross-sectional profile. In some instances, at least some of the elongate members 114 may be fibers that are formed of a biosorbable material. At least some of the elongate members 114 may be formed of a biosorbable polymer. At least some of the elongate members 114 may be formed of a biosorbable metal. As an example, at least some of the elongate members 114 may be magnesium fibers. As another example, at least some of the elongate members 114 may be elastic fibers. In some cases, at least some of the elongate members 114 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the elongate members 114 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. In some cases, at least some of the magnesium fibers may have a length that is greater than the overall length of the bio-compatible implant 100 such that at least some of the magnesium fibers extend beyond the periphery 108 of the bio-compatible implant 100. In some cases, the magnesium fibers that extend beyond the periphery 108 of the bio-compatible implant 100 may be used to help anchor the bio-compatible implant 100 at the treatment site by engaging one or more of the magnesium fibers with a bone anchor such as a bone staple. Other anchoring devices are also contemplated.

In instances in which at least some of the elongate members 114 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 100. In some cases, at least some of the elastin fibers may have a length that is greater than the overall length of the bio-compatible implant 100 such that at least some of the elastin fibers extend beyond the periphery 108 of the bio-compatible implant 100. In some cases, the elastin fibers that extend beyond the periphery 108 of the bio-compatible implant 100 may be used to help anchor the bio-compatible implant 100 at the treatment site by engaging one or more of the elastin fibers with a bone anchor such as a bone staple. Other anchoring devices are also contemplated.

In instances in which at least some of the elongate members 114 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 100. In some cases, at least some of the high density, highly cross-linked collagen fibers may have a length that is greater than the overall length of the bio-compatible implant 100 such that at least some of the high density, highly cross-linked collagen fibers extend beyond the periphery 108 of the bio-compatible implant 100. In some cases, the high density, highly cross-linked collagen fibers that extend beyond the periphery 108 of the bio-compatible implant 100 may be used to help anchor the bio-compatible implant 100 at the treatment site by engaging one or more of the high density, highly cross-linked collagen fibers with a bone anchor such as a bone staple. Other anchoring devices are also contemplated.

In FIG. 6C, the reinforcing element 110 includes a structure 116 that is disposed within the collagen scaffold 102. In some instances, the structure 116 includes a number of elongate members 118 that are connected together via connecting links 120. The connecting links 120 may be fibers that are wrapped around the elongate members 118 in order to hold the structure 116 together while the collagen scaffold 102 is formed around the structure 116, for example. In some instances, the connecting links 120 may be surgical suture material. The connecting links 120 may be formed of the same material as used to form the elongate members 118. In some instances, the structure 116 may represent a unitary molded structure.

The elongate members 118 may take any of a variety of different forms. In some cases, as shown, each of the elongate members 118 may have a random cross-sectional profile. In some instances, at least some of the elongate members 118 may be fibers that are formed of a biosorbable material. At least some of the elongate members 118 may be formed of a biosorbable polymer. At least some of the elongate members 118 may be formed of a biosorbable metal. As an example, at least some of the elongate members 118 may be magnesium fibers. As another example, at least some of the elongate members 118 may be elastic fibers. In some cases, at least some of the elongate members 118 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the elongate members 118 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers may have a length that is greater than the overall length of the bio-compatible implant 100 such that at least some of the magnesium fibers extend beyond the periphery 108 of the bio-compatible implant 100. In some cases, the magnesium fibers that extend beyond the periphery 108 of the bio-compatible implant 100 may be used to help anchor the bio-compatible implant 100 at the treatment site by engaging one or more of the magnesium fibers with a bone anchor such as a bone staple. Other anchoring devices are also contemplated.

In instances in which at least some of the elongate members 118 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 100. In some cases, at least some of the elastin fibers may have a length that is greater than the overall length of the bio-compatible implant 100 such that at least some of the elastin fibers extend beyond the periphery 108 of the bio-compatible implant 100. In some cases, the elastin fibers that extend beyond the periphery 108 of the bio-compatible implant 100 may be used to help anchor the bio-compatible implant 100 at the treatment site by engaging one or more of the elastin fibers with a bone anchor such as a bone staple. Other anchoring devices are also contemplated.

In instances in which at least some of the elongate members 118 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 100. In some cases, at least some of the high density, highly cross-linked collagen fibers may have a length that is greater than the overall length of the bio-compatible implant 100 such that at least some of the high density, highly cross-linked collagen fibers extend beyond the periphery 108 of the bio-compatible implant 100. In some cases, the high density, highly cross-linked collagen fibers that extend beyond the periphery 108 of the bio-compatible implant 100 may be used to help anchor the bio-compatible implant 100 at the treatment site by engaging one or more of the high density, highly cross-linked collagen fibers with a bone anchor such as a bone staple. Other anchoring devices are also contemplated.

In FIG. 6D, the reinforcing element 110 includes a structure 120 that is disposed within the collagen scaffold 102. In some instances, the structure 120 includes a number of elongate members 122 that are joined together prior to forming the collagen scaffold 102 around the structure 120. In some instances, at least some of the elongate members 122 may be secured together, such as adhesively, prior to encapsulating the structure 120 within the collagen scaffold 102.

The elongate members 120 may take any of a variety of different forms. In some cases, as shown, each of the elongate members 120 may have a random cross-sectional profile. In some instances, at least some of the elongate members 120 may be fibers that are formed of a biosorbable material. At least some of the elongate members 120 may be formed of a biosorbable polymer. At least some of the elongate members 120 may be formed of a biosorbable metal. As an example, at least some of the elongate members 120 may be magnesium fibers. As another example, at least some of the elongate members 120 may be elastic fibers. In some cases, at least some of the elongate members 120 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the elongate members 120 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers may have a length that is greater than the overall length of the bio-compatible implant 100 such that at least some of the magnesium fibers extend beyond the periphery 108 of the bio-compatible implant 100. In some cases, the magnesium fibers that extend beyond the periphery 108 of the bio-compatible implant 100 may be used to help anchor the bio-compatible implant 100 at the treatment site by engaging one or more of the magnesium fibers with a bone anchor such as a bone staple. Other anchoring devices are also contemplated.

In instances in which at least some of the elongate members 120 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 100. In some cases, at least some of the elastin fibers may have a length that is greater than the overall length of the bio-compatible implant 100 such that at least some of the elastin fibers extend beyond the periphery 108 of the bio-compatible implant 100. In some cases, the elastin fibers that extend beyond the periphery 108 of the bio-compatible implant 100 may be used to help anchor the bio-compatible implant 100 at the treatment site by engaging one or more of the elastin fibers with a bone anchor such as a bone staple. Other anchoring devices are also contemplated.

In instances in which at least some of the elongate members 120 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 100. In some cases, at least some of the high density, highly cross-linked collagen fibers may have a length that is greater than the overall length of the bio-compatible implant 100 such that at least some of the high density, highly cross-linked collagen fibers extend beyond the periphery 108 of the bio-compatible implant 100. In some cases, the high density, highly cross-linked collagen fibers that extend beyond the periphery 108 of the bio-compatible implant 100 may be used to help anchor the bio-compatible implant 100 at the treatment site by engaging one or more of the high density, highly cross-linked collagen fibers with a bone anchor such as a bone staple. Other anchoring devices are also contemplated.

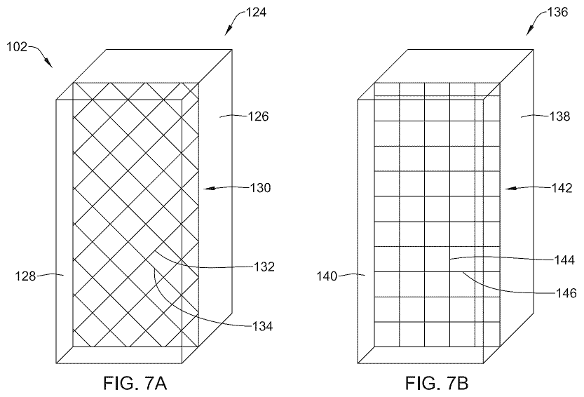

FIG. 7A shows an illustrative bio-compatible implant 124. The bio-compatible implant 124 may be used for any of a variety of soft tissue repairs, such as but not limited to the Gluteus Medius, which is a large fan-shaped muscle located in the posterior hip, the Hip Capsule, which is also in the hip. The bio-compatible implant 124 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 124 may be used in treating soft tissue injuries in the Achilles tendon. The bio-compatible implant 124 may be used in treating soft tissue injuries for any areas of the body that will accommodate the bio-compatible implant 124.

The bio-compatible implant 124 may be considered as including the scaffold 102. While drawn as rectilinear, it will be appreciated that this is merely illustrative, as the bio-compatible implant 124 may take any desired shape to best fit at a desired treatment site such as but not limited to a rotator cuff repair. In some cases, the bio-compatible implant 124 may be largely rectilinear, with rounded over corners, for example. In some cases, the bio-compatible implant 124 may be at least partially ovoid. In some cases, the bio-compatible implant 124 may curve around a treatment site. These are just examples.

As shown, the collagen scaffold 102 includes a first collagen layer 126 and a second collagen layer 128. Sandwiched between the first collagen layer 126 and the second collagen layer 128 is a reinforcing layer 130. In some instances, the reinforcing layer 130 has a cross-hatch pattern, formed by a first plurality of reinforcing elements 132 extending in a first direction forming an acute angle with a longitudinal or load axis of the bio-compatible implant 124 and a second plurality of reinforcing elements 134 extending in a second direction, different from the first direction, forming an acute angle with the longitudinal or load axis of the bio-compatible implant 124. In some cases, the first plurality of reinforcing elements 132 meet the second plurality of reinforcing elements 134 at a right angle. In some instances, the first plurality of reinforcing elements 132 may meet the second plurality of reinforcing elements 134 at an acute angle that is less than 90 degrees.

The reinforcing layer 130 may be formed of a variety of different materials. In some cases, the reinforcing layer 130 may include magnesium fibers, elastin fibers, and high density, highly cross-linked collagen fibers. In some instances, the reinforcing layer 130 may be formed of silk. The reinforcing layer 130 may include chitosan, for example. When the reinforcing layer 130 is formed of, or otherwise includes silk, the silk fibers may have a length of at least 50 millimeters or more, and an average diameter in a range of 10 to 13 microns as a single fiber. In some cases, multiple silk fibers can be woven together to form a larger composite fiber. In some cases, multiple silk fibers may be formed into a silk textile layer. The reinforcing layer 130 may be formed by braiding or knitting together two or more silk fibers, for example.

In some cases, the reinforcing layer 130 is completely encapsulated within the collagen scaffold 102. In some instances, at least some of the fibers forming the reinforcing layer 130 may extend beyond a periphery of the bio-compatible implant 124 such that at least some of the fibers may be adapted to engage with a bone anchor such as a bone staple. In some instances, the reinforcing layer 130 may be considered as being porous, and the first collagen layer 126 and/or the second collagen layer 128 may each extend at least partially into the reinforcing layer 130. In some cases, the reinforcing layer 130 may provide the bio-compatible implant 124 with an initial tensile modulus of 6 to 12 mega Pascals (MPa) and an ultimate tensile strength of at least about 50 Newtons (N). In some case, the bio-compatible implant 124 may have an ultimate tensile strength in the range of 50 N to 1,000 N, in the range of 50 N to 500 N, in the range of 50 N to 300 N, or in the range of 50 N to 250 N. In some cases, the bio-compatible implant 124 may have an ultimate tensile strength of as high as about 5000 N, depending on the native strength of the tendon being repaired.

FIG. 7B shows an illustrative bio-compatible implant 136. The bio-compatible implant 136 may be used for any of a variety of soft tissue repairs, such as but not limited to the Gluteus Medius, which is a large fan-shaped muscle located in the posterior hip, the Hip Capsule, which is also in the hip. The bio-compatible implant 136 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 136 may be used in treating soft tissue injuries in the Achilles tendon. The bio-compatible implant 136 may be used in treating soft tissue injuries for any areas of the body that will accommodate the bio-compatible implant 136.

The bio-compatible implant 136 may be considered as including the scaffold 102. While drawn as rectilinear, it will be appreciated that this is merely illustrative, as the bio-compatible implant 136 may take any desired shape to best fit at a desired treatment site such as but not limited to a rotator cuff repair. In some cases, the bio-compatible implant 136 may be largely rectilinear, with rounded over corners, for example. In some cases, the bio-compatible implant 136 may be at least partially ovoid. In some cases, the bio-compatible implant 136 may curve around a treatment site. These are just examples.

As shown, the collagen scaffold 102 includes a first collagen layer 138 and a second collagen layer 140. Sandwiched between the first collagen layer 138 and the second collagen layer 140 is a reinforcing layer 142. In some instances, the reinforcing layer 142 has a cross-hatch pattern, formed by a first plurality of reinforcing elements 144 extending in a first direction parallel or at least substantially parallel with a longitudinal or load axis of the bio-compatible implant 136 and a second plurality of reinforcing elements 146 extending a second direction perpendicular or at least substantially perpendicular with the longitudinal or load axis of the bio-compatible implant 136.

The reinforcing layer 142 may be formed of a variety of different materials. In some cases, the reinforcing layer 142 may include magnesium fibers, elastin fibers, and high density, highly cross-linked collagen fibers. In some instances, the reinforcing layer 142 may be formed of silk. The reinforcing layer 142 may include chitosan, for example. When the reinforcing layer 142 is formed of, or otherwise includes silk, the silk fibers may have a length of at least 50 millimeters or more, and an average diameter in a range of 10 to 13 microns as a single fiber. In some cases, multiple silk fibers can be woven together to form a larger composite fiber. In some cases, multiple silk fibers may be formed into a silk textile layer. The reinforcing layer 130 may be formed by braiding or knitting together two or more silk fibers, for example.

In some cases, the reinforcing layer 142 is completely encapsulated within the collagen scaffold 102. In some instances, at least some of the fibers forming the reinforcing layer 142 may extend beyond a periphery of the bio-compatible implant 124 such that at least some of the fibers may be adapted to engage with a bone anchor such as a bone staple. In some instances, the reinforcing layer 142 may be considered as being porous, and the first collagen layer 126 and/or the second collagen layer 128 may each extend at least partially into the reinforcing layer 130.

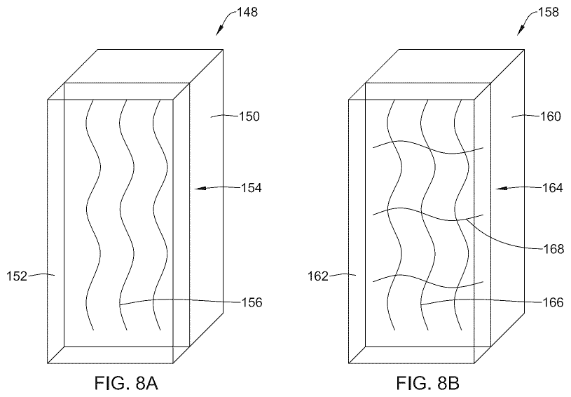

FIG. 8A shows an illustrative bio-compatible implant 148. The bio-compatible implant 148 may be used for any of a variety of soft tissue repairs, such as but not limited to the Gluteus Medius, which is a large fan-shaped muscle located in the posterior hip, the Hip Capsule, which is also in the hip. The bio-compatible implant 148 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 148 may be used in treating soft tissue injuries in the Achilles tendon. The bio-compatible implant 148 may be used in treating soft tissue injuries for any areas of the body that will accommodate the bio-compatible implant 148.

The bio-compatible implant 148 may be considered as including the scaffold 102. While drawn as rectilinear, it will be appreciated that this is merely illustrative, as the bio-compatible implant 148 may take any desired shape to best fit at a desired treatment site such as but not limited to a rotator cuff repair. In some cases, the bio-compatible implant 148 may be largely rectilinear, with rounded over corners, for example. In some cases, the bio-compatible implant 148 may be at least partially ovoid. In some cases, the bio-compatible implant 148 may curve around a treatment site. These are just examples.

As shown, the collagen scaffold 102 includes a first collagen layer 150 and a second collagen layer 152. Sandwiched between the first collagen layer 148 and the second collagen layer 150 is a reinforcing layer 154. In some instances, the reinforcing layer 154 includes a number of individual fibers 156 extending in a direction at least substantially parallel with a longitudinal or load axis of the bio-compatible implant 148. While the individual fibers 156 are shown as being substantially co-planar, this is not required in all cases. In some instances, at least some of the individual fibers 156 may extend at least partially into the first collagen layer 150. In some instances, at least some of the individual fibers 156 may extend at least partially into the second collagen layer 152.

The individual fibers 156 may take any of a variety of different forms. In some instances, at least some of the individual fibers 156 may be formed of a biosorbable material. At least some of the individual fibers 156 may be formed of a biosorbable polymer. At least some of the individual fibers 156 may be formed of a biosorbable metal. As an example, at least some of the individual fibers 156 may be magnesium fibers. As another example, at least some of the individual fibers 156 may be elastic fibers. In some cases, at least some of the individual fibers 156 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the individual fibers 156 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers may have a length that is greater than the overall length of the bio-compatible implant 100 such that at least some of the magnesium fibers extend beyond the periphery 108 of the bio-compatible implant 100. In some cases, the magnesium fibers that extend beyond the periphery 108 of the bio-compatible implant 100 may be used to help anchor the bio-compatible implant 100 at the treatment site by engaging one or more of the magnesium fibers with a bone anchor such as a bone staple. Other anchoring devices are also contemplated.

In instances in which at least some of the individual fibers 156 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 100. In some cases, at least some of the elastin fibers may have a length that is greater than the overall length of the bio-compatible implant 100 such that at least some of the elastin fibers extend beyond the periphery 108 of the bio-compatible implant 100. In some cases, the elastin fibers that extend beyond the periphery 108 of the bio-compatible implant 100 may be used to help anchor the bio-compatible implant 100 at the treatment site by engaging one or more of the elastin fibers with a bone anchor such as a bone staple. Other anchoring devices are also contemplated.

In instances in which at least some of the individual fibers 156 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 100. In some cases, at least some of the high density, highly cross-linked collagen fibers may have a length that is greater than the overall length of the bio-compatible implant 100 such that at least some of the high density, highly cross-linked collagen fibers extend beyond the periphery 108 of the bio-compatible implant 100. In some cases, the high density, highly cross-linked collagen fibers that extend beyond the periphery 108 of the bio-compatible implant 100 may be used to help anchor the bio-compatible implant 100 at the treatment site by engaging one or more of the high density, highly cross-linked collagen fibers with a bone anchor such as a bone staple. Other anchoring devices are also contemplated.

In some cases, the reinforcing layer 154 is completely encapsulated within the collagen scaffold 102. In some instances, at least some of the fibers forming the reinforcing layer 130 may extend beyond a periphery of the bio-compatible implant 124 such that at least some of the fibers may be adapted to engage with a bone anchor such as a bone staple. In some instances, the reinforcing layer 154 may be considered as being porous, and the first collagen layer 150 and/or the second collagen layer 152 may each extend at least partially into the reinforcing layer 154.

FIG. 8B shows an illustrative bio-compatible implant 158. The bio-compatible implant 158 may be used for any of a variety of soft tissue repairs, such as but not limited to the Gluteus Medius, which is a large fan-shaped muscle located in the posterior hip, the Hip Capsule, which is also in the hip. The bio-compatible implant 158 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 158 may be used in treating soft tissue injuries in the Achilles tendon. The bio-compatible implant 158 may be used in treating soft tissue injuries for any areas of the body that will accommodate the bio-compatible implant 158.

The bio-compatible implant 158 may be considered as including the scaffold 102. While drawn as rectilinear, it will be appreciated that this is merely illustrative, as the bio-compatible implant 158 may take any desired shape to best fit at a desired treatment site such as but not limited to a rotator cuff repair. In some cases, the bio-compatible implant 158 may be largely rectilinear, with rounded over corners, for example. In some cases, the bio-compatible implant 158 may be at least partially ovoid. In some cases, the bio-compatible implant 158 may curve around a treatment site. These are just examples.

As shown, the collagen scaffold 102 includes a first collagen layer 160 and a second collagen layer 162. Sandwiched between the first collagen layer 160 and the second collagen layer 162 is a reinforcing layer 164. In some instances, the reinforcing layer 164 includes a number of individual fibers 166 extending in a direction at least substantially parallel with a longitudinal or load axis of the bio-compatible implant 158. The reinforcing layer 164 may, as shown, include a number of tie fibers 168 extending in a direction at least substantially perpendicular with the longitudinal or load axis of the bio-compatible implant 158. While the individual fibers 166 and 168 are shown as being substantially co-planar, this is not required in all cases. In some instances, at least some of the individual fibers 166 and 168 may extend at least partially into the first collagen layer 160. In some instances, at least some of the individual fibers 166 and 168 may extend at least partially into the second collagen layer 162.

The individual fibers 166 and 168 may take any of a variety of different forms. In some instances, at least some of the individual fibers 166 and 168 may be formed of a biosorbable material. At least some of the individual fibers 166 and 168 may be formed of a biosorbable polymer. At least some of the individual fibers 166 and 168 may be formed of a biosorbable metal. As an example, at least some of the individual fibers 166 and 168 may be magnesium fibers. As another example, at least some of the individual fibers 166 and 168 may be elastic fibers. In some cases, at least some of the individual fibers 166 and 168 may be high-density, highly cross-linked collagen fibers, dispersed within the collagen scaffold 102. In contrast, the collagen scaffold 102 may otherwise be formed of a random network of dispersed collagen fibers that are not high density and that are not highly cross-linked.

In instances in which at least some of the individual fibers 166 and 168 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers, particularly those forming the individual fibers 166, may have a length that is greater than the overall length of the bio-compatible implant 100 such that at least some of the magnesium fibers extend beyond a periphery of the bio-compatible implant 148. In some cases, the magnesium fibers that extend beyond the periphery 108 of the bio-compatible implant 148 may be used to help anchor the bio-compatible implant 148 at the treatment site by engaging one or more of the magnesium fibers with a bone anchor such as a bone staple. Other anchoring devices are also contemplated.

In instances in which at least some of the individual fibers 166 and 168 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 148. In some cases, at least some of the elastin fibers may have a length that is greater than the overall length of the bio-compatible implant 100 such that at least some of the elastin fibers extend beyond the periphery 108 of the bio-compatible implant 148. In some cases, the elastin fibers that extend beyond the periphery 108 of the bio-compatible implant 148 may be used to help anchor the bio-compatible implant 148 at the treatment site by engaging one or more of the elastin fibers with a bone anchor such as a bone staple. Other anchoring devices are also contemplated.

In instances in which at least some of the individual fibers 166 and 168 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 148. In some cases, at least some of the high density, highly cross-linked collagen fibers may have a length that is greater than the overall length of the bio-compatible implant 148 such that at least some of the high density, highly cross-linked collagen fibers extend beyond a periphery of the bio-compatible implant 148. In some cases, the high density, highly cross-linked collagen fibers that extend beyond the periphery of the bio-compatible implant 148 may be used to help anchor the bio-compatible implant 148 at the treatment site by engaging one or more of the high density, highly cross-linked collagen fibers with a bone anchor such as a bone staple. Other anchoring devices are also contemplated.

In some cases, the reinforcing layer 164 is completely encapsulated within the collagen scaffold 102. In some instances, at least some of the fibers forming the reinforcing layer 164 may extend beyond a periphery of the bio-compatible implant 124 such that at least some of the fibers may be adapted to engage with a bone anchor such as a bone staple. In some instances, the reinforcing layer 164 may be considered as being porous, and the first collagen layer 160 and/or the second collagen layer 162 may each extend at least partially into the reinforcing layer 164.

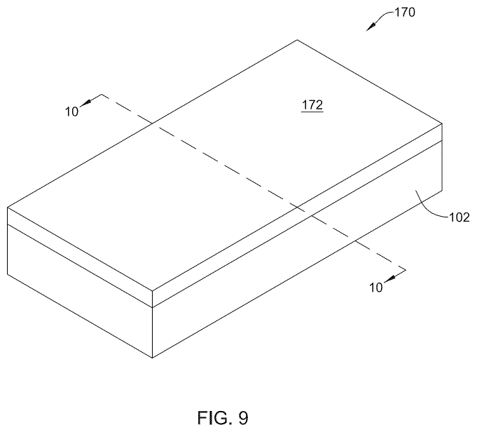

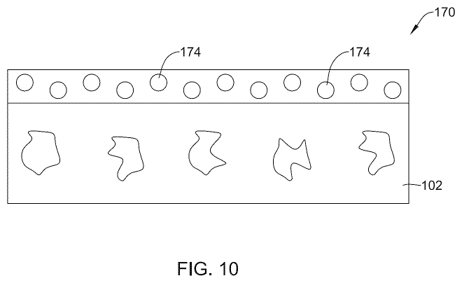

FIG. 9 shows a schematic diagram of an example bio-compatible implant 170 and FIG. 10 is a cross-sectional view thereof, taken along the line 10-10 in FIG. 9. The bio-compatible implant 170 may be used for any of a variety of soft tissue repairs, such as but not limited to the Gluteus Medius, which is a large fan-shaped muscle located in the posterior hip, the Hip Capsule, which is also in the hip. The bio-compatible implant 170 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 170 may be used in treating soft tissue injuries in the Achilles tendon. The bio-compatible implant 170 may be used in treating soft tissue injuries for any areas of the body that will accommodate the bio-compatible implant 170.

For purposes of illustration, the bio-compatible implant 170 will be described herein with respect to rotator cuff repairs. The bio-compatible implant 170 may be considered as being an example of the implant 12 discussed with respect to FIGS. 1-2, 3A-3C and 4. The bio-compatible implant 170 may be considered as including the scaffold 102. While drawn as rectilinear, it will be appreciated that this is merely illustrative, as the bio-compatible implant 170 may take any desired shape to best fit at a desired treatment site such as but not limited to a rotator cuff repair. In some cases, the bio-compatible implant 170 may be largely rectilinear, with rounded over corners, for example. In some cases, the bio-compatible implant 170 may be at least partially ovoid. In some cases, the bio-compatible implant 170 may curve around a treatment site. These are just examples.

The bio-compatible implant 170 includes a reinforcing sheet 172 that is secured to the collagen scaffold 102. In some cases, the reinforcing sheet 172 may be adhesively secured to the collagen scaffold 102, for example. The reinforcing sheet 172 may be considered as providing additional strength to the bio-compatible implant 170 immediately after implantation, before any tissue ingrowth into the collagen substrate 102 has occurred. In some instances, the reinforcing sheet 172 may be biosorbable.

As seen for example in FIG. 10, the reinforcing sheet 172 includes a number of reinforcing fibers 174 that extend longitudinally within the reinforcing sheet 172, along a load axis of the bio-compatible implant 170. The reinforcing fibers 174 may include magnesium fibers, for example. In some instances, the reinforcing fibers 174 may include elastin fibers. The reinforcing fibers 174 may include collagen fibers. For example, the reinforcing fibers 174 may include high density, highly cross-linked collagen fibers.

In instances in which at least some of the reinforcing fibers 174 are magnesium fibers, the magnesium fibers may have average diameters that are in a range of 0.1 to 1.5 microns or more. At least some of the magnesium fibers may have a circular cross-sectional profile, as shown. In some cases, at least some of the magnesium fibers may have a less-defined cross-sectional profile. The magnesium fibers may have lengths that are as long or even longer than an overall length of the collagen scaffold 102, depending on whether the magnesium fibers are arranged longitudinally or are dispersed in multiple directions. As an example, the magnesium fibers may have lengths that range from about 1 millimeter to about 50 millimeters or more. In some cases, the magnesium fibers may be woven into a mesh or in a back-and-forth configuration. In some cases, the magnesium may be present as a thin sheet. The magnesium fibers may have an average length that is less than an overall length of the bio-compatible implant 100. In some cases, at least some of the magnesium fibers, particularly those forming the individual fibers 166, may have a length that is greater than the overall length of the bio-compatible implant 100 such that at least some of the magnesium fibers extend beyond a periphery of the bio-compatible implant 148. In some cases, the magnesium fibers that extend beyond the periphery 108 of the bio-compatible implant 148 may be used to help anchor the bio-compatible implant 148 at the treatment site by engaging one or more of the magnesium fibers with a bone anchor such as a bone staple. Other anchoring devices are also contemplated.

In instances in which at least some of the reinforcing fibers 174 are elastin fibers, elastin fibers may have average diameters that are in a range of 0.1 microns to 1.5 microns. At least some of the elastin fibers may have a circular cross-sectional profile, as shown. In some cases, at least some of the elastin fibers may have a less-defined cross-sectional profile. In some cases, elastin fibers may sprawl in both a longitudinal direction and in a radial direction within the collagen scaffold 102. The elastin fibers may have lengths that range from 10 microns to 500 microns. The elastin fibers may have an average length that is less than an overall length of the bio-compatible implant 148. In some cases, at least some of the elastin fibers may have a length that is greater than the overall length of the bio-compatible implant 100 such that at least some of the elastin fibers extend beyond the periphery 108 of the bio-compatible implant 148. In some cases, the elastin fibers that extend beyond the periphery 108 of the bio-compatible implant 148 may be used to help anchor the bio-compatible implant 148 at the treatment site by engaging one or more of the elastin fibers with a bone anchor such as a bone staple. Other anchoring devices are also contemplated.

In instances in which at least some of the reinforcing fibers 174 are high density, highly cross-linked collagen fibers, the high density, highly cross-linked collagen fibers may have average diameters that are in a range of 0.1 microns to 3 microns. At least some of the high density, highly cross-linked collagen fibers may have a circular cross-sectional profile, as shown. In some cases, at least some of the high density, highly cross-linked collagen fibers may have a less-defined cross-sectional profile. In some cases, the high density, highly cross-linked collagen fibers may sprawl in both a longitudinal direction and in a radial direction within the collagen scaffold 102. The high density, highly cross-linked collagen fibers may have lengths that range from 10 microns to 100 microns. The high density, highly cross-linked collagen fibers may have an average length that is less than an overall length of the bio-compatible implant 170. In some cases, at least some of the high density, highly cross-linked collagen fibers may have a length that is greater than the overall length of the bio-compatible implant 170 such that at least some of the high density, highly cross-linked collagen fibers extend beyond a periphery of the bio-compatible implant 170. In some cases, the high density, highly cross-linked collagen fibers that extend beyond the periphery of the bio-compatible implant 170 may be used to help anchor the bio-compatible implant 170 at the treatment site by engaging one or more of the high density, highly cross-linked collagen fibers with a bone anchor such as a bone staple. Other anchoring devices are also contemplated.

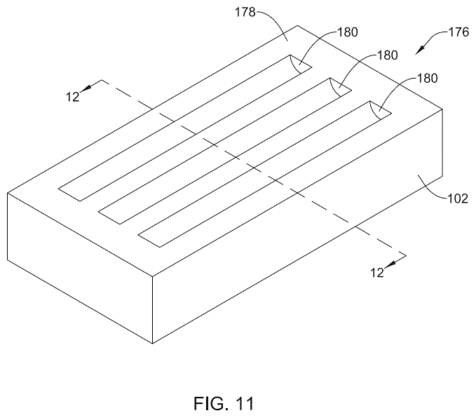

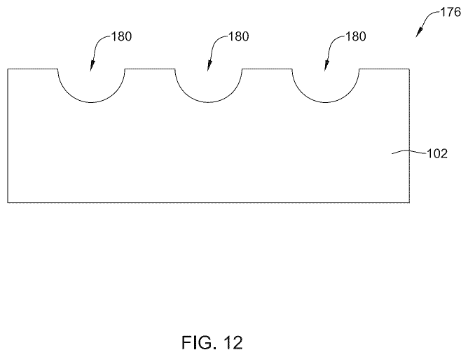

FIG. 11 shows a schematic diagram of an example bio-compatible implant 176 and FIG. 12 is a cross-sectional view thereof, taken along the line 12-12 in FIG. 11. The bio-compatible implant 176 may be used for any of a variety of soft tissue repairs, such as but not limited to the Gluteus Medius, which is a large fan-shaped muscle located in the posterior hip, the Hip Capsule, which is also in the hip. The bio-compatible implant 176 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 176 may be used in treating soft tissue injuries in the Achilles tendon. The bio-compatible implant 176 may be used in treating soft tissue injuries for any areas of the body that will accommodate the bio-compatible implant 176.

For purposes of illustration, the bio-compatible implant 176 will be described herein with respect to rotator cuff repairs. The bio-compatible implant 176 may be considered as being an example of the implant 12 discussed with respect to FIGS. 1-2, 3A-3C and 4. The bio-compatible implant 176 may be considered as including the scaffold 102. While drawn as rectilinear, it will be appreciated that this is merely illustrative, as the bio-compatible implant 176 may take any desired shape to best fit at a desired treatment site such as but not limited to a rotator cuff repair. In some cases, the bio-compatible implant 176 may be largely rectilinear, with rounded over corners, for example. In some cases, the bio-compatible implant 176 may be at least partially ovoid. In some cases, the bio-compatible implant 176 may curve around a treatment site. These are just examples.

The bio-compatible implant 176 includes the collagen scaffold 102. As shown in FIG. 11, the bio-compatible implant 176 includes an upper surface 178, with a number of longitudinally-extending voids 180 disposed within the upper surface 178. While “upper” is a relative term, in this case “upper” may refer to a surface of the bio-compatible implant 176 that is positioned at an uppermost orientation when the bio-compatible implant 176 is implanted. Put another way, the upper surface 178 may be considered as being a surface of the bio-compatible implant 176 that is still accessible once the bio-compatible implant 176 has been implanted.

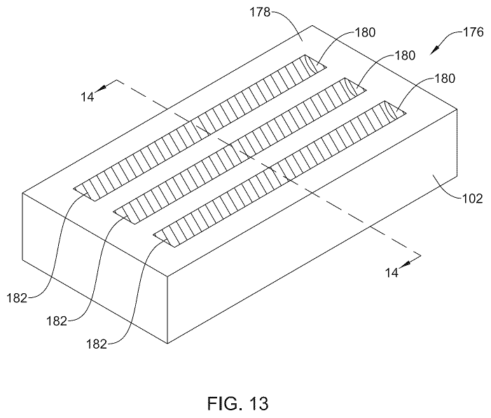

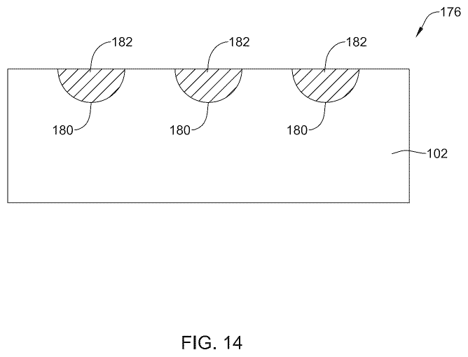

The voids 180 may be formed in any manner, including molding the voids 180 into the collagen scaffold 102 when the collagen scaffold 102 is formed. In some cases, the voids 180 may be ground or cut into the upper surface 178 of the bio-compatible implant 176. The voids 180 may extend a substantial length of the upper surface 178, for example. After the bio-compatible implant 176 has been implanted, the voids 180 may be filled with a strengthening agent. The strengthening agent may be a liquid when applied to the voids 180, for example, and may cure or otherwise dry into a solid that functions as a reinforcing member. FIG. 13 shows the bio-compatible implant 176 after the voids 180 have been filled with a strengthening agent 182 while FIG. 14 is a cross-sectional view taken along the line 14-14 of FIG. 13. In some cases, the strengthening agent 182 may be a fibrin glue that is applied as a liquid, and then cures into a solid that functions as a reinforcing member.

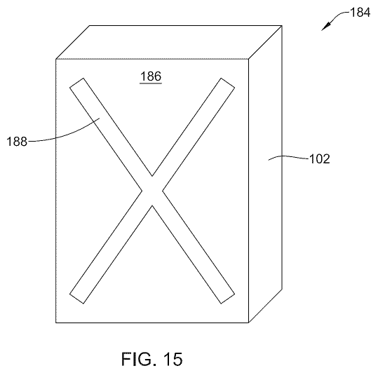

In FIGS. 11 to 14, the voids 180 are shown as extending in parallel with a longitudinal or load axis of the bio-compatible implant 176. In some instances, the voids 180 may also have a radial component. FIG. 15 shows a bio-compatible implant 184 that includes the collagen scaffold 102. As shown, the bio-compatible implant 184 has a top surface 186 having an “X” shaped void 186 formed within the top surface 186. The “X-shaped void 186 can subsequently be filled with a strengthening agent in the same manner as the voids 180 shown in FIGS. 11 to 14.

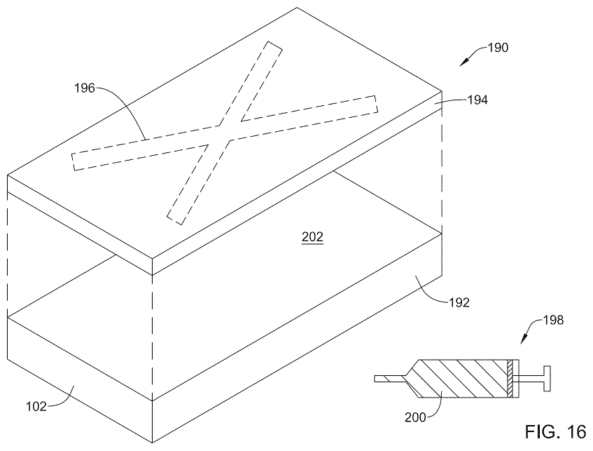

FIG. 16 shows a kit 190 that may be used in a soft tissue repair. The kit 190 includes a bio-compatible implant 192 including the collagen scaffold 102. Instead of forming or otherwise including voids (such as the voids 180 and 188) within a surface of the bio-compatible implant 192, the kit 190 includes a mold 194 that may be temporarily placed atop the bio-compatible implant 192 after the bio-compatible implant 192 has been implanted in order to guide placement of a strengthening agent. The bio-compatible implant 192 may be formed of just collagen, for example. In some cases, the bio-compatible implant 192 may additionally include any of the strengthening elements described with respect to the bio-compatible implant 100, 124, 136, 148 and 158, for example.

The mold 194, which in some cases may be formed of polytetrafluoroethylene (PTFE), also known as TEFLON®, includes a cutout 196 that provides a guide for where the strengthening agent should be placed. It will be appreciated that the mold 194 may include any variety of shapes for the cutout 196, and the “X” shaped cutout 196 is merely illustrative. The kit 190 includes a syringe 198 that contains a strengthening agent 200. The syringe 198 may be used to fill the cutout 196 within the mold 194 with the strengthening agent 200. Once the strengthening agent 200 has at least partially cured within the cutout 196, the mold 194 may be removed, leaving the hardening strengthening agent 200 on an upper surface 202. The strengthening agent 200, which may be biosorbable, provides additional strength to the bio-compatible implant 192 before tissue ingrowth begins.

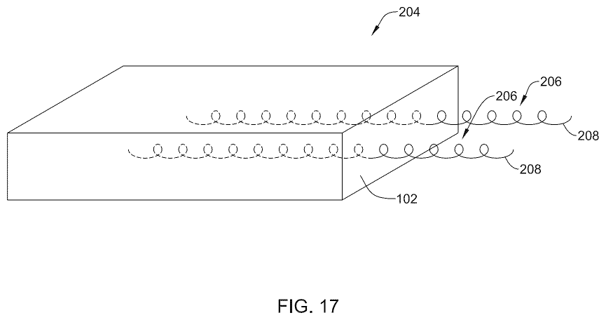

FIG. 17 shows a schematic diagram of an example bio-compatible implant 204. The bio-compatible implant 204 may be used for any of a variety of soft tissue repairs, such as but not limited to the Gluteus Medius, which is a large fan-shaped muscle located in the posterior hip, the Hip Capsule, which is also in the hip. The bio-compatible implant 204 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 204 may be used in treating soft tissue injuries in the Achilles tendon. The bio-compatible implant 204 may be used in treating soft tissue injuries for any areas of the body that will accommodate the bio-compatible implant 204.

For purposes of illustration, the bio-compatible implant 204 will be described herein with respect to rotator cuff repairs. The bio-compatible implant 204 may be considered as being an example of the implant 12 discussed with respect to FIGS. 1-2, 3A-3C and 4. The bio-compatible implant 204 may be considered as including the scaffold 102. While drawn as rectilinear, it will be appreciated that this is merely illustrative, as the bio-compatible implant 204 may take any desired shape to best fit at a desired treatment site such as but not limited to a rotator cuff repair. In some cases, the bio-compatible implant 204 may be largely rectilinear, with rounded over corners, for example. In some cases, the bio-compatible implant 204 may be at least partially ovoid. In some cases, the bio-compatible implant 204 may curve around a treatment site. These are just examples.

The bio-compatible implant 204 includes one or more coiled elements 206 that extend into the collagen scaffold 102. In some cases, the coiled elements 206 may be molded into the collagen scaffold 102. In some cases, the collagen scaffold 102 may be formed without including the coiled elements 206, and the coiled elements 206 may be subsequently threaded into the collagen scaffold 102. The coiled elements 206 may extend far enough out of the collagen scaffold 102 to be able to be used to help secure the bio-compatible implant 204 in place at the treatment site by using one or more bone anchors, such as bone staples, to secure a free end 208 of each of the coiled elements 206 in place. While the coiled elements 206 are shown as only extending past one end of the bio-compatible implant 204, this is merely illustrative, as in some instances the coiled elements 206 may be long enough to extend out both ends of the bio-compatible implant 204.

The coiled elements 206 may be formed of any suitable polymeric or metallic material. In some cases, the coiled elements 206 may be formed of a biosorbable polymeric or metallic material. As an example, the coiled elements 206 may be formed of magnesium, although other materials are contemplated as well. In some cases, the coiled elements 206 may be configured to allow for some change in length in response to applied forces.

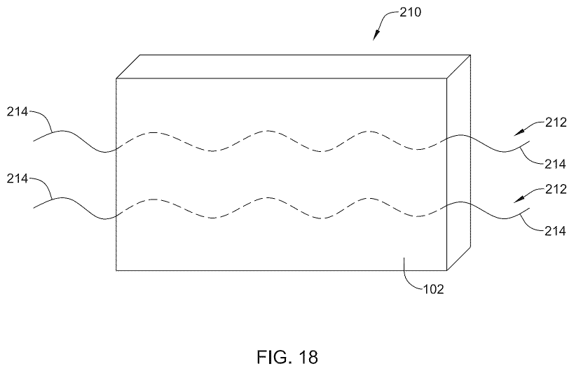

FIG. 18 shows a schematic diagram of an example bio-compatible implant 210. The bio-compatible implant 210 may be used for any of a variety of soft tissue repairs, such as but not limited to the Gluteus Medius, which is a large fan-shaped muscle located in the posterior hip, the Hip Capsule, which is also in the hip. The bio-compatible implant 210 may be used in treating soft tissue injuries in the knee, such as but not limited to ligaments such as the ACL (anterior cruciate ligament), MCL (medial collateral ligament) and the PCL (posterior cruciate ligament) and tendons such as the hamstring tendons, the quadriceps tendon and the patellar tendon. The bio-compatible implant 210 may be used in treating soft tissue injuries in the Achilles tendon. The bio-compatible implant 210 may be used in treating soft tissue injuries for any areas of the body that will accommodate the bio-compatible implant 210.

For purposes of illustration, the bio-compatible implant 210 will be described herein with respect to rotator cuff repairs. The bio-compatible implant 210 may be considered as being an example of the implant 12 discussed with respect to FIGS. 1-2, 3A-3C and 4. The bio-compatible implant 210 may be considered as including the scaffold 102. While drawn as rectilinear, it will be appreciated that this is merely illustrative, as the bio-compatible implant 204 may take any desired shape to best fit at a desired treatment site such as but not limited to a rotator cuff repair. In some cases, the bio-compatible implant 210 may be largely rectilinear, with rounded over corners, for example. In some cases, the bio-compatible implant 210 may be at least partially ovoid. In some cases, the bio-compatible implant 210 may curve around a treatment site. These are just examples.

The bio-compatible implant 210 includes one or more filaments, such as wave elements 212, that extend into the collagen scaffold 102. In some cases, the filaments (e.g., wave elements 212) may be molded into the collagen scaffold 102. In some cases, the collagen scaffold 102 may be formed without including the filaments (e.g., wave elements 212), and the filaments (e.g., wave elements 212) may be subsequently threaded into the collagen scaffold 102. The filaments (e.g., wave elements 212) may extend far enough out of the collagen scaffold 102 to be able to be used to help secure the bio-compatible implant 210 in place at the treatment site by using one or more bone anchors, such as bone staples, to secure a free end 214 of each of the filaments (e.g., wave elements 212) in place.

The filaments (e.g., wave elements 212) may be formed of any suitable polymeric or metallic material. In some cases, the filaments (e.g., wave elements 212) may be formed of a biosorbable polymeric or metallic material. As an example, the filaments (e.g., wave elements 212) may be formed of magnesium, although other materials are contemplated as well. In some cases, the filaments (e.g., wave elements 212) may be configured to allow for some change in length in response to applied forces.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A bio-compatible implant adapted for use in repairing soft tissue damage, the bio-compatible implant comprising:
a collagen scaffold comprising a plurality of non-woven reconstituted collagen fibers; and
a reinforcing layer coupled with the collagen scaffold, the reinforcing layer comprising a plurality of reinforcing fibers extending longitudinally within the reinforcing layer along a load axis of the bio-compatible implant.

2. The bio-compatible implant of claim 1, wherein the collagen scaffold includes a first collagen layer and a second collagen layer, with the reinforcing layer sandwiched between the first collagen layer and the second collagen layer.

3. The bio-compatible implant of claim 2, wherein the first collagen layer and/or the second collagen layer extends into the reinforcing layer.

4. The bio-compatible implant of claim 2, wherein the reinforcing fibers include a first plurality of reinforcing elements extending in a first direction substantially parallel with the load axis of the bio-compatible implant and a second plurality of reinforcing elements extending in a second direction substantially perpendicular with the load axis of the bio-compatible implant.

5. The bio-compatible implant of claim 4, wherein at least some of the reinforcing fibers extend beyond a periphery of the collagen scaffold.

6. The bio-compatible implant of claim 4, wherein the bio-compatible implant has an overall length of 25 to 35 millimeters, an overall width of 20 to 30 millimeters, and an overall thickness of 1 to 3 millimeters.

7. The bio-compatible implant of any claim 4, wherein the reinforcing fibers are encapsulated within the collagen scaffold by forming the collagen scaffold around the reinforcing fibers.

8. The bio-compatible implant of claim 4, wherein the plurality of reinforcing fibers are formed of a biosorbable polymer.

9. The bio-compatible implant of claim 1, further comprising a plurality of tie fibers extending through the reinforcing layer, at least some of the plurality of tie fibers at least substantially non-parallel with the load axis of the bio-compatible implant.

10. The bio-compatible implant of claim 1, wherein the reinforcing layer further comprises a plurality of collagen fibers.

11. The bio-compatible implant of claim 10, wherein the reinforcing layer further comprises a plurality of high-density, highly cross-linked collagen fibers.

12. The bio-compatible implant of claim 1, wherein at least some of the reinforcing fibers comprise magnesium fibers.

13. The bio-compatible implant of claim 12, wherein the magnesium fibers have an average length up to about 50 millimeters and/or an average diameter in a range of 0.1 microns to 1.5 microns.

14. The bio-compatible implant of claim 1, wherein at least some of the reinforcing fibers comprise elastin fibers randomly dispersed within the collagen scaffold.

15. The bio-compatible implant of claim 14, wherein the elastin fibers have an average length in a range of 10 microns to 500 microns and/or an average diameter in a range of 0.1 microns to 1.5 microns.

16. A bio-compatible implant adapted for use in repairing soft tissue damage, the bio-compatible implant comprising:
a collagen scaffold comprising a plurality of non-woven reconstituted collagen fibers, the collagen scaffold including a first collagen layer and a second collagen layer; and
a reinforcing layer sandwiched between the first collagen layer and the second collagen layer, the reinforcing layer comprising a plurality of reinforcing fibers formed of a biosorbable polymer;
wherein the plurality of reinforcing fibers include a first plurality of reinforcing fibers extending in a first direction substantially parallel with a load axis of the bio-compatible implant and a second plurality of reinforcing fibers extending in a second direction substantially perpendicular with the load axis of the bio-compatible implant; and
wherein the first plurality of reinforcing fibers extend into the reinforcing layer.

17. The bio-compatible implant of claim 16, wherein the second plurality of reinforcing fibers extend into the reinforcing layer.

18. The bio-compatible implant of claim 16, wherein the collagen scaffold has a porosity of at least 80 percent or more.

19. The bio-compatible implant of claim 16, wherein the bio-compatible implant has an overall length of 25 to 35 millimeters, an overall width of 20 to 30 millimeters, and an overall thickness of 1 to 3 millimeters.

20. The bio-compatible implant of claim 16, wherein the first plurality of reinforcing fibers and the second plurality of reinforcing fibers form a fiber mat.
